(19) United States
(12) Patent Application Publication
(10) Pub. No.: US 2021/0386352 A1
(43) Pub. Date: Dec. 16, 2021

(54) BIOLOGICAL SIGNAL MONITORING WEAR

(71) Applicant: TORAY INDUSTRIES, INC., Tokyo (JP)

(72) Inventors: Ichiro ITAGAKI, Tokyo (JP); Hiromi TAKARADA, Tokyo (JP); Ryo MATSUO, Tokyo (JP)

(73) Assignee: TORAY INDUSTRIES, INC., Tokyo (JP)

(21) Appl. No.: 17/284,282

(22) PCT Filed: Sep. 20, 2019
(86) PCT No.: PCT/JP2019/037064

§ 371 Date: Apr. 9, 2021

(30) Foreign Application Priority Data
Oct. 12, 2018 (JP) 2018-193713

(51) Int. Cl. A61B 5/332; A61B 5/256; A61B 5/282; A61B 5/00

(52) U.S. Cl. CPC A61B 5/332; A61B 5/7221; A61B 5/282; A61B 5/256

(57) ABSTRACT

A biological signal monitoring wear includes: a biological signal measurement device; two or more electrodes contacting to skin; and a wear main portion in which an electrically conductive body configured to connect between the biological signal measurement device and the electrodes is supported on a cloth member of the wear main portion, and an elastic body having a length in a range of 30% to 60% inclusive relative to a length around a trunk in a subject's solar plexus portion is fixed to a trunk portion in the cloth member.

### FIELD

The present invention relates to a wear to monitor a biological signal such as an electrocardiogram for a long period of time, particularly to a biological signal monitoring wear to diagnose an atrial fibrillation and an irregular pulse by recording an electrocardiogram in an environment of a normal daily life.

### BACKGROUND

In the heart, an electrical excitation that is generated in a sinus node is transmitted to an atria muscle thereby causing shrinkage of an atria. The electrical excitation in the atria is transmitted to an atrioventricular node and then to an atria muscle through a special myocardium called “cardiac conduction system” such as a His bundle and a Purkinje fiber, thereby causing shrinkage of a ventricle, in turn, resulting in pulsation. The electrocardiogram that is widely used in diagnosis is waveform data in which active electric potential waves of 7 cardiac conduction systems starting from the sinus node are overlapped, so that waveform information relating to an active electric potential in the heart is included. When a rhythm of the heart is disturbed, an interval between the pulsations becomes irregular, and then, this appears in the electrocardiogram as an “irregular pulse”. In the case of “cardiac infarction” or “angina attack”, the electrical excitation of the myocardium is disturbed thereby resulting in an abnormal waveform in the electrocardiogram. Also, when there is damage such as an inflammation in the myocardium, an abnormal electrocardiogram is recognized.

In the heart diseases such as an angina and an irregular pulse, the abnormality is not always recognized in the electrocardiogram. The abnormality appears in the electrocardiogram only when the stroke occurs. Therefore, when the stroke does not occur, the electrocardiogram cannot be distinguished at all from the normal electrocardiogram. Because of this, a test is carried out by measurement of the electrocardiogram under an exercise load and in a normal daily life for a long period of time. In general, as the electrocardiogram test for a long period of time, a 24-hour Holter electrocardiogram test is widely carried out. Domestically, the number of the test with this method reaches 1.5 million on yearly basis. Although this electrocardiogram test is widely used, it is pointed that the heart diseases cannot be always found even with the electrocardiogram test for 24 hours, so that an electrocardiogram test for a long period time such as 1 week or longer is preferable. In particular, diagnosis of asymptomatic atrial fibrillation, which is a cause of a cerebral infarction, is difficult; and thus, a detection rate of the fibrillation by the 24-hour Holter electrocardiogram test is only about 1% to 5%. An implantable loop recorder, which requires an implanting operation under the skin, can detect a low-frequency atrial fibrillation by continuing recording of the electrocardiogram for a long period of time; but this is an invasive and expensive test method, and insurance coverage of the test is limited only to the cerebral infarction and syncope with unknown causes. As for a non-invasive electrocardiogram test, it is reported that the atrial fibrillation can be effectively detected by an external loop recorder with an automatic irregular pulse detector. But it is pointed out that this has problems in poor quality of the electrocardiogram and a high quasi-positive ratio due to algorithm, as compared with the usual Holter electrocardiogram.

As for the method to measure the electrocardiogram comfortably and conveniently under an environment of a normal daily life, the use of a so-called wearable biological signal monitoring system with an electrode and a measuring device attached to a cloth or a belt has been attempted. In order to monitor the biological signal for a long period of time such as 1 week or longer, it is important that this system can be taken off and put off upon taking a bath or the like, and that a sensor such as the electrode can be readily positioned even by a subject not having a specialized knowledge, and that stable information with a low noise can be obtained so that the disease can be diagnosed with a method like an electrocardiogram analysis. To fulfil these requirements, many inventions of the wear incorporating a sensor such as the electrode have been made. Hereinafter, typical examples of these inventions and the problems thereof will be described.

Patent Literature 1 discloses a wearable electrode having a fibrous structural electrode formed of a nanofiber and an electrically conductive polymer to enhance an adhesion with the skin, and a woven fabric that can suppress the movement of the electrode portion even when the wear to which the electrode is attached moves due to the body movement of a subject. The electrode formed of the nanofiber is not only excellent in adhesion to the skin but also high in hydrophilicity; thus, this has a characteristic that a stable biological signal can be obtained during the time of a body movement even if a force obtained from the wear is small and thereby a pressure applied to the electrode is low. However, it is difficult to provide the wear that completely matches to the size of each individual subject; and thus, in the case of the subject having a shorter length around a trunk in the subject's solar plexus portion than a standard wear size, the force obtained is so weak that it is difficult to obtain a biological signal in a level enough to analyze the electrocardiogram. On the other hand, when the force of the wear is too strong, an excessive pressure is applied to the subject thereby causing an uncomfortable feeling to the subject.

Patent Literature 2 discloses a wear in which silicone rubber or the like that is excellent in an anti-slipping property is arranged around a sensor. It is described that in this wear because the silicone rubber coheres to the skin, a stable biological signal can be obtained even during a sport exercise without using an adhesive. Although a Holter electrocardiogram testing instrument using this invention has already been on the market in Europe, the content described in this patent literature cannot be realized; therefore, in order to obtain a stable electrocardiogram, a paste having an electric conductivity needs to be applied onto the electrode surface to enhance an adhesion thereof to the skin. To apply the paste onto the electrode surface at every putting-on and taking-off of the wear is cumbersome to the subject. In addition, a highly adhesive paste can cause not only an uncomfortable feeling such as an itchy skin but also a skin trouble.

Patent Literature 3 is the invention relating to a biological signal monitoring wear having a band-like structure that is adjustable in accordance with a size and a body shape of the subject. In this invention, characteristics of an elastic body used for the band are not described. In general, the elastic body in the band-like form has a problem of temporal deterioration. Therefore, even when an expansion rate thereof is kept constant, the force obtained therefrom decreases with passage of time. Accordingly, unless the elastic body having a low temporal deterioration in this force is selected, in many cases a stable biological signal that can be used in a clinical test cannot be obtained in about 3 days of a measurement period. Besides, in this invention, a specific method to apply a constant electrode pressure to the skin of the subjects having different sizes is not described. According to the content that is disclosed in this patent literature, because a suitable pressure cannot be applied to the electrode, the pressure applied to the electrode is so low that a sufficient level of a biological signal for the electrocardiogram analysis cannot be obtained, or the pressure is so high that it gives an uncomfortable feeling to the subject.

As explained above, in the known technical field, in order to obtain a stable biological signal with a low noise even during the time of a body movement, a pressure applied to a sensor such as the electrode is important; and thus, it can be concluded that there is a need to develop a biological signal monitoring wear having a mechanism to supply a suitable and constant pressure to a sensor such as the electrode for a long period of time in accordance with various body shapes and sizes of the subjects.

### CITATION LIST

### Patent Literature

Patent Literature 1: Japanese Patent Application Laid-open No. 2017-527510

Patent Literature 2: Japanese Patent No. 5707504

Patent Literature 3: National Publication of International Patent Application No. 2009-518057

### SUMMARY

### Technical Problem

An object of the present invention is to provide a wear that can detect a stable signal with a low noise thereby enabling to diagnose a disease comfortably and conveniently with an electrocardiogram analysis or the like for a long period of time such as 1 week or longer in subjects having various body shapes and sizes and spending a normal daily life including walking, and ascending and descending of a staircase.

### Solution to Problem

The present inventors have completed the present invention as a result of repeated diligent research in order to solve the above problems. A biological signal monitoring wear according to the present invention includes: a biological signal measurement device; two or more electrodes contacting to skin; and a wear main portion in which an electrically conductive body configured to connect between the biological signal measurement device and the electrodes is supported on a cloth member of the wear main portion, and an elastic body having a length in a range of 30% to 60% inclusive relative to a length around a trunk in a subject's solar plexus portion is fixed to a trunk portion in the cloth member.

In the biological signal monitoring wear according to the present invention, a force to expand the elastic body by 30% in a longitudinal direction of the elastic body is in a range of 3 N to 9 N inclusive.

In the biological signal monitoring wear according to the present invention, a force to expand the elastic body by 20% in a longitudinal direction of the elastic body is in a range of 2 N to 6 N inclusive.

In the biological signal monitoring wear according to the present invention, when the elastic body is stored for 10 days under a normal temperature and humidity condition with an expansion rate of the elastic body kept at 30% in a longitudinal direction of the elastic body, a force to expand the elastic body by 10% after the storage is 80% or more relative to the force required before the storage.

The biological signal monitoring wear according to the present invention further includes a size-adjustment functioning portion to adjust an expansion rate of the elastic body in a subject having a different length around a trunk in a solar plexus portion.

In the biological signal monitoring wear according to the present invention, the size-adjustment functioning portion has tick marks in a size-adjustment portion in the size-adjustment functioning portion.

In the biological signal monitoring wear according to the present invention, the wear main portion comprises a front body, a back body, and at least one shoulder strap configured to connect between the front body and the back body.

In the biological signal monitoring wear according to the present invention, the front body and the back body are a bib-type wear in which at least one side (side of the body) of the bib-type wear is cut and separated to each other.

In the biological signal monitoring wear according to the present invention, the electrodes each is an electrically conductive fiber.

In the biological signal monitoring wear according to the present invention, the electrodes are each formed of a nanofiber whose fiber diameter is in a range of 10 nm to 5000 nm inclusive.

In the biological signal monitoring wear according to the present invention, the electrodes each comprise an electrically conductive sheet whose adhesion strength measured with a 90-degree peel-off method in accordance with JIS-Z0237 is 200 g/20 mm or less.

### Advantageous Effects of Invention

In the biological signal monitoring wear according to the present invention, an elastic body is fixed to a cloth member having an electrode, an electric wiring, and a measuring device arranged in respective prescribed locations and thereby a pressure is applied stably and appropriately to the electrode that is contacting to the skin; by so doing, a stable signal with a low noise can be detected so that a disease can be diagnosed comfortably and conveniently with an electrocardiogram analysis or the like for a long period of time such as 1 week or longer in subjects having various body shapes and sizes and spending a normal daily life including walking, and ascending and descending of a staircase.

### BRIEF DESCRIPTION OF DRAWINGS

FIG. 1 is a drawing of the biological signal monitoring wear according to an embodiment of the present invention viewed from a front right of the subject who puts on this wear.

FIG. 2 is a drawing of a back side (the side contacting to the skin) of a front body of the biological signal monitoring wear according to an embodiment of the present invention.

FIG. 3 is a back-side drawing viewed from a rear left of the subject who puts on the biological signal monitoring wear according to an embodiment of the present invention.

FIG. 4 is a back-side drawing of the biological signal monitoring wear according to an embodiment of the present invention.

FIG. 5 is a front view of the biological signal monitoring wear according to an embodiment of the present invention.

FIG. 6 is an enlarged drawing of a size-adjustment functioning portion in the front body of the biological signal monitoring wear according to an embodiment of the present invention.

FIG. 7 includes drawings illustrating stress-strain curves before storage (FIG. 7(A)) and after storage (FIG. 7(B)) of an elastic body that is suitable for the biological signal monitoring wear according to an embodiment of the present invention.

FIG. 8 includes drawings illustrating stress-strain curves before storage (FIG. 8(A)) and after storage (FIG. 8(B)) of an elastic body that is unsuitable for the biological signal monitoring wear according to an embodiment of the present invention.

FIG. 9 is an electrocardiogram during the time of body movement obtained in Comparative Example 1.

FIG. 10 is an electrocardiogram during the time of body movement obtained in Example 1.

FIG. 11 is an electrocardiogram during the time of body movement obtained in Comparative Example 2.

FIG. 12 is an electrocardiogram when wearing for 2 weeks obtained in Example 2.

FIG. 13 is a drawing illustrating a summary portion in the electrocardiogram analysis report obtained in Example 3.

FIG. 14 is a drawing illustrating part of a registered waveform in the electrocardiogram analysis report obtained in Example 3.

FIG. 15 is a drawing illustrating part of a compressed waveform in the electrocardiogram analysis report obtained in Example 3.

### DESCRIPTION OF EMBODIMENTS

Hereinafter, the biological signal monitoring wear according to the present invention will be explained in detail on the basis of these drawings. It must be noted here that the present invention is not restricted by these embodiments.

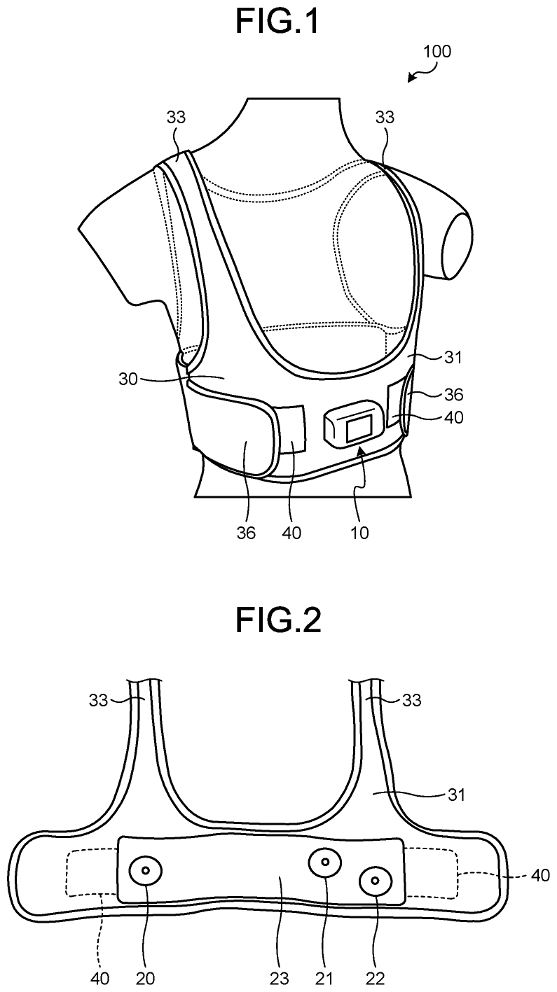

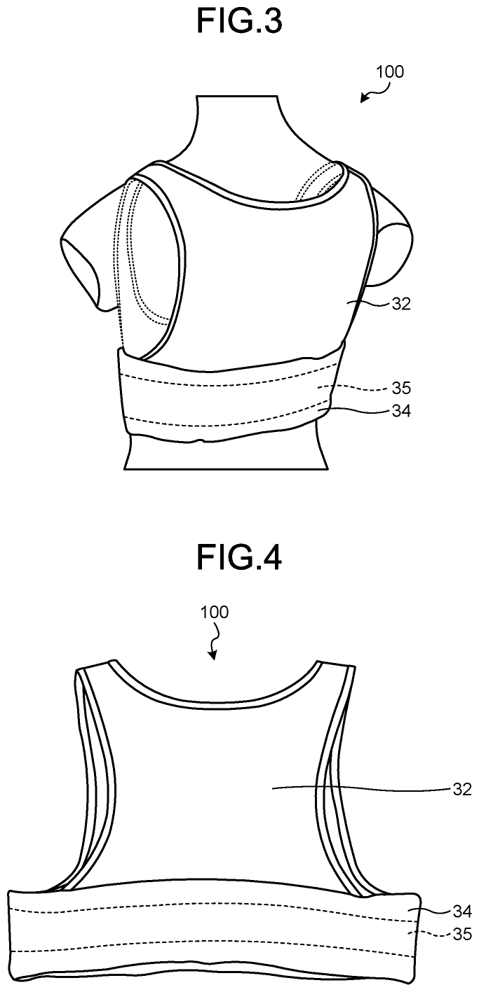

FIG. 1 is a drawing of a biological signal monitoring wear 100 according to an embodiment of the present invention viewed from a front right of the subject who puts on this wear. FIG. 2 is a drawing of a back side (the side contacting to the skin) of a front body of the biological signal monitoring wear 100. FIG. 3 is a back-side drawing of the biological signal monitoring wear 100 according to an embodiment of the present invention viewed from a rear left of the subject who puts on this wear. FIG. 4 is a back-side drawing of the biological signal monitoring wear 100. The biological signal monitoring wear 100 has an electrocardiograph 10, which is a measurement device of the biological signal, electrodes 20, 21, and 22, as well as a wear main portion 30.

The wear main portion 30 has a front body 31 and a back body 32; the front body 31 and the back body 32 are connected only by two shoulder straps 33; and the front body 31 and the back body 32 are cut and separated to each other in both sides (sides of the body). When the front body 31 and the back body 32 are cut and separated to each other in at least one side (side of the body), the biological signal monitoring wear 100 can be readily put on. Although it is preferable that the front body 31 and the back body 32 be cut and separated to each other in both sides (sides of the body), the front body 31 and the back body 32 may be connected in both sides. When the front body 31 and the back body 32 are connected by at least one of the shoulder straps 33, the wear main portion 30 can be prevented from positional displacement.

The electrocardiograph 10, which is the biological signal measurement device, is attached to a center of a trunk portion in the front body 31. As illustrated in FIG. 2, in the back side of the trunk portion in the front body 31, i.e., the portion to which the electrocardiograph 10 is attached, the electrodes 20, 21, and 22, which contacts to the subject's skin, are fixed. These electrodes 20, 21, and 22 are arranged in accordance with CCS, which is one of the induction methods of the Holter electrocardiogram. The electrode 20 serves as a plus electrode, the electrode 21 serves as a minus electrode, and the electrode 22 serves as an earth electrode. Although not illustrated in the drawings, each of the electrodes 20, 21, and 22 is connected to a connector 37 (see FIG. 6) of the electrocardiograph 10 through lead wires. To cover these electric wiring portions, an electrically insulating member 23 is used.

In the biological signal monitoring wear 100 according to the present invention, the electrodes 20, 21, and 22, with which the biological signal is detected from a body, are formed of an electrically conductive fiber. Preferably, the electrically conductive fiber is a fibrous structural body that is impregnated with an electrically conductive polymer. More preferably, the fibrous structural body is formed of multi-filaments, and the electrically conductive polymer is supported onto the fiber surface and into a void created between the monofilaments that constitute the fibrous structural body.

There is no particular restriction in the electrically conductive polymer used in the electrodes 20, 21, and 22 according to the present invention so far as this polymer is a resin having an electric conductivity. Illustrative examples thereof include electrically conductive polymers such as PEDOT/PSS and an electrically conductive resin composition blended with carbon black, carbon nanotube (CNT), metal particulate, or the like. Use of a resin having an elastic property such as an elastomer resin is not preferable because stable detection of the signal is difficult due to a change in the electric conductivity depending on an elastic condition. In view of safety and processability, the electrically conductive polymer to be used in the electrodes 20, 21, and 22 is more preferably PEDOT/PSS in which the resin itself is an electrically conductive polymer having the electric conductivity. Here, PEDOT/PSS is the PEDOT, which is a thiophene type electrically conductive polymer, doped with polystyrene sulfonic acid (poly(4-styrene sulfonate) (PSS)).

Illustrative examples of the form of the fibrous structural body to be used for the electrodes 20, 21, and 22 include: textile-like bodies such as a knitted body, a woven body, and an unwoven cloth; and a strap-like body. Preferably, a knitted body and a woven body are used.

Materials of the fiber to be used in the fibrous structural body according to the present invention are synthetic fibers and the like. Illustrative examples of the synthetic fiber include: fibers formed of polyethylene terephthalate, polypropylene terephthalate, or polybutylene terephthalate; aromatic polyester type fibers formed by copolymerizing these polymers with a third component; aliphatic polyester type fibers represented by those formed of L-lactic acid as a main component therein; polyamide type fibers such as nylon 6 and nylon 66; acryl fibers formed of polyacrylonitrile as a main component therein; polyolefin type fibers such as polyethylene and polypropylene; and polyvinyl chloride type fibers. In addition, a fiber blended with an additive such as titanium oxide, and a fiber having a polymer that is reformed so as to be provided with functionality such as an enhanced moisture-absorption property may also be used.

From a viewpoint to support the electrically conductive resin onto a fiber surface and into a void created between fibers, it is preferable that the fibrous structural body according to the present invention include multi-filaments having monofilaments whose fiber diameter is 0.2 dtex or less. The mixing rate of the multi-filaments having monofilaments with the size of 0.2 dtex or less in the fibrous structural body is not particularly restricted so far as the performance thereof is not affected. In view of electric conductivity and durability, preferably the mixing rate is higher, and more preferably the rate is in the range of 50% to 100% inclusive. Also, the more the number of the monofilament is, the more the electrically conductive resin is supported in the fibrous structural body, because the void formed of plurality of the monofilaments, i.e., the portion in which the electrically conductive resin is supported, is subdivided. In addition, even if the void is subdivided due to the reduced fiber diameter, continuity of the electrically conductive resin can be retained; and thus, superior high electric conductivity and washing durability can be obtained. A microfiber whose fiber diameter is 5 μm or less, the size used in an artificial leather, an outer material, or the like, is preferable; a nanofiber whose fiber diameter is in the range of 10 nm to 5000 nm inclusive is more preferable.

The fibrous structural body including, as the nanofiber, nanofibers produced by a known method, such as a nanofiber staple yarn aggregate produced from “Nanoalloy (registered trade mark)” fiber and a monofilament yarn aggregate produced by an electrospinning method or the like may be preferably used, although the fibrous structural body including a nanofiber multi-filament yarn is more preferable. The nanofiber multi-filament yarn may be produced by a known conjugate spinning method or the like. Among others, for example, a nanofiber multi-filament yarn having a small fluctuation in the fiber diameter thereof that is obtained by removing a sea portion of a conjugate fiber using a conjugate spinneret may be effectively used (this is illustrated in Japanese Patent Application Laid-open No. 2013-185283); but the nanofiber multi-filament yarn is not limited to these.

The electrodes 20, 21, and 22 to be used in the present invention are not limited to the electrically conductive fiber; an electrically conductive sheet having an adhesion property including an electrically conductive material may be used. When this electrically conductive sheet is used for the electrodes 20, 21, and 22 according to the present invention, the adhesion strength of the electrically conductive sheet measured with a 90-degree peel-off method in accordance with JIS-Z0237 is preferably 200 g/20 mm or less.

The size and shape of the electrodes 20, 21, and 22 are not particularly specified so far as the biological signal can be detected. Both the vertical and horizontal lengths thereof are preferably in the range of 2.0 cm to 20.0 cm inclusive. Illustrative examples of the electrodes 20, 21, and 22 that can be used include “hitoe” Medical Electrode and “hitoe” Medical Electrode II (both are manufactured by Toray Medical Co., Ltd.).

Preferably, the electrocardiograph 10 used in the biological signal monitoring wear 100 according to the present invention is put on, taken off from, and connected to the wear main portion 30 by the connector 37 (see FIG. 6). When the electrocardiograph 10 is taken off from the wear main portion 30, this main portion can be washed. There is no particular restriction in the connector 37, and a socket or the like that is generally used to connect to an electric code may be used, although it is more preferable to use a plurality of metal dot buttons that can fix the electrocardiograph 10 to the wear main portion 30 at the same time.

When the electrocardiograph 10 is electrically charged in advance, the electrocardiograph has a function to memorize the electrocardiogram date for a period of 2 weeks or longer without being charged. It is more preferable that this have a function to transfer the data to a mobile terminal or to a personal computer by communication. With this function, it becomes possible to take and store the data, for example, into the personal computer, thereby enabling to readily analyze the data.

In the biological signal monitoring wear 100 according to the present invention, lead wires are necessary to transfer the biological signals obtained by the electrodes 20, 21, and 22 to the electrocardiograph 10. Preferably, the lead wire is formed by a method in which the electrically conductive resin is printed to the wear main portion 30, or a method in which a film of the electrically conductive resin is laminated to this main portion, or the wire may be formed of a fiber having electric conductivity or a metal wire.

When the lead wire is formed of a fiber having electric conductivity, this electrically conductive fiber may be a yarn in which a polyester fiber or a nylon fiber is covered with a metal fiber including silver, aluminum, or stainless steel, or an electrically conductive fiber in which carbon black is composite-arranged in part of a core or a shell of polyester or nylon in a longitudinal direction of the fiber, or a metal-coated yarn in which a polyester fiber or a nylon fiber is coated with a metal including silver, aluminum, or stainless steel. In view of durability and versatility, the use of the yarn in which a polyester fiber or a nylon fiber is covered with a metal fiber including silver, aluminum, or stainless steel is especially preferable. The lead wire such as “hitoe” Medical Lead Wire or “hitoe” Medical Lead Wire II, both being manufactured by Toray Medical Co., Ltd., may be used.

The lead wire formed by printing or like of the electrically conductive fiber or of the electrically conductive resin is covered with the electrically insulating member 23 having a width of 50 mm. A polyurethane type water-proof seam tape manufactured by Toray Coatex Co., Ltd. (E502; manufactured by Toray Coatex Co., Ltd.) or the like may be used as the electrically insulating member 23.

A preferable method to attach the electrically conductive fiber used in the lead wire to the wear main portion 30 is as follows. Namely, the lead wire formed of an electrically conductive tape obtained by weaving the electrically conductive fiber into a belt-like shape is interposed between the cloth of the wear main portion 30 and the electrically insulating member 23 having an electrical insulating property and being provided with a hot-melt adhesive on one side thereof, and then adhered by heating. To both ends of the lead wire, electrically conductive snap buttons are disposed across the insulating sheet provided with a hot-melt adhesive. The electrodes 20, 21, and 22 and the connector 37 of the electrocardiograph 10 are connected to each of the snap buttons.

In the wear main portion 30 in the biological signal monitoring wear 100 according to the present invention, a two-way tricot or a smooth knit, which are used in an underwear or the like, may be used. A material having good elasticity is preferable for the cloth thereof, and a material that sufficiently absorbs sweat and is comfortable in a skin-contact feeling is more preferable. Illustrative examples of the material usable therein include polyester type synthetic fibers such as polyethylene terephthalate, polytrimethylene terephthalate, and polybutylene terephthalate, as well as polyamide type synthetic fibers such as nylon. In addition, cotton and hemp may also be used as a natural material.

In a trunk portion 34 in the back body 32, a flat rubber having a width of 40 mm is incorporated as an elastic body 35. Polyurethane or a natural rubber is used as the rubber material of the flat rubber. The length of the elastic body 35 is in the range of 30 to 60% inclusive relative to the length around the trunk in the subject's solar plexus portion. When the length of the elastic body 35 is in the range of 30% to 60% inclusive relative to the length around the trunk in the subject's solar plexus portion, the biological signal can be obtained at a proper pressure of the electrodes 20, 21, and 22, which are supported to the wear main portion 30, to the subject's skin, namely without a strong pressure felt by the subject upon wearing. The width of the elastic body 35 is preferably in the range of about 25 mm to 50 mm.

The force to expand the elastic body 35 by 30% in the longitudinal direction thereof is preferably in the range of 3 N to 9 N inclusive. When the force to expand the elastic body 35 by 30% in the longitudinal direction thereof is less than 3 N, the pressure to the subject's skin is so low that there is a risk that it may be difficult to obtain the biological signal. When the force to expand the elastic body 35 by 30% in the longitudinal direction thereof is more than 9 N, the pressure felt by the subject is so high that the comfortability upon wearing this deteriorates.

The force to expand the elastic body 35 by 20% in the longitudinal direction thereof is preferably in the range of 2 N to 6 N inclusive. Illustrative examples of the elastic body 35 usable therein include LY-40 (manufactured by Kitani Co., Ltd.).

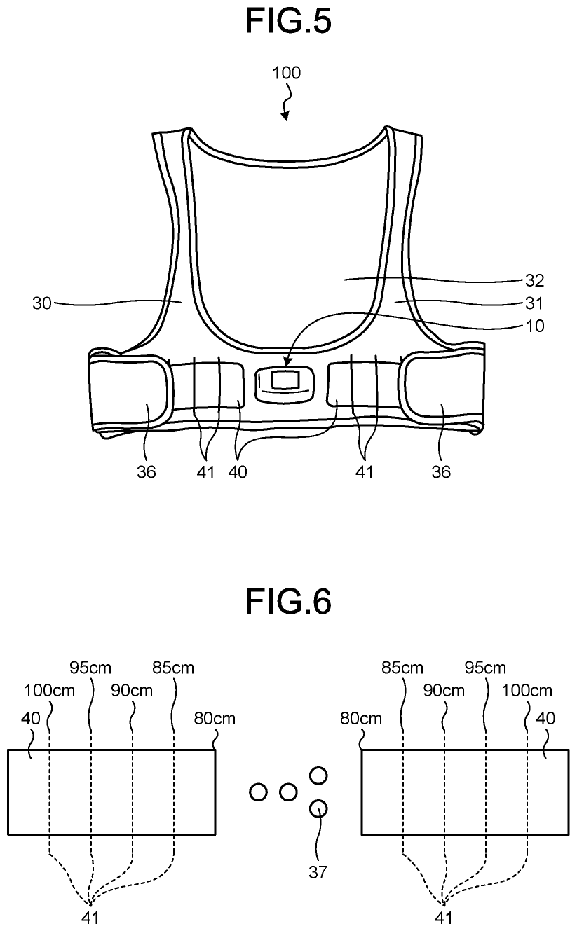

FIG. 5 is a front view of the biological signal monitoring wear 100. To the trunk portion of the front body 31, a B-face (loop face) 40 of a plane fastener is sewn in such a way that an expansion rate of the flat rubber that is incorporated to the back body 32 may be evenly fixed in accordance with the size of the length around the trunk in the wearer's solar plexus portion. In both ends of the trunk portion in the back body 32, side tabs 36 are located, and are fixed to the B-face 40 of the plane fastener of the front body 31 by an A-face (hook face) of a plane fastener that is arranged in the backside of the side tab 36 so as to connect the front body 31 with the back body 32 in both sides (sides of the body). The A-face of the plane fastener and the B-face 40 of the plane fastener function as a size-adjustment functioning portion. In order to make easy to find a holding position of the side tab 36, stitches 41 are formed as tick marks on the B-face 40 of the plane fastener. The stitches 41 are stitched with a color thread (interval of 2.5 cm) so as to be easily recognized.

FIG. 6 is an enlarged drawing of the B-face 40 of the plane fastener, which is a size-adjustment functioning portion in the front body 31 of the biological signal monitoring wear 100. FIG. 6 illustrates the M-size wear that is used to the size of the length in the range of 80 cm to 100 cm around the trunk in the subject's solar plexus portion. For example, when the size of the length around the trunk in the subject's solar plexus portion is 90 cm, the right and left side tabs 36 are held in such a way that the front ends thereof may be held at the second positions of the stitches 41 from the connector 37 of the electrocardiograph 10. When the size of the length around the trunk in the solar plexus portion is 87 cm, the side tabs 36 are held in such a way that the front ends thereof may be held at the position moved about 1 cm toward the second stitch 41 from the position of the first stitch 41, or at the position slightly moved to the first stitch 41 from a halfway between the first stitch 41 and the second stitch 41. Here, it is preferable to mark the holding position with an oily marker so as not to forget the holding position.

In the elastic body 35 used in the present invention, when the elastic body 35 is stored for 10 days under normal temperature and humidity condition with the expansion rate thereof kept at 30% in the longitudinal direction thereof, the force to expand the elastic body 35 by 10% is preferably 80% or more relative to the force required before the storage. This is because in the elastic body 35, the electrodes 20, 21, and 22 need to be contacted to the subject's skin by applying a constant pressure to the skin for 1 week or longer; therefore, a change in the shear-strain relation required to be small.

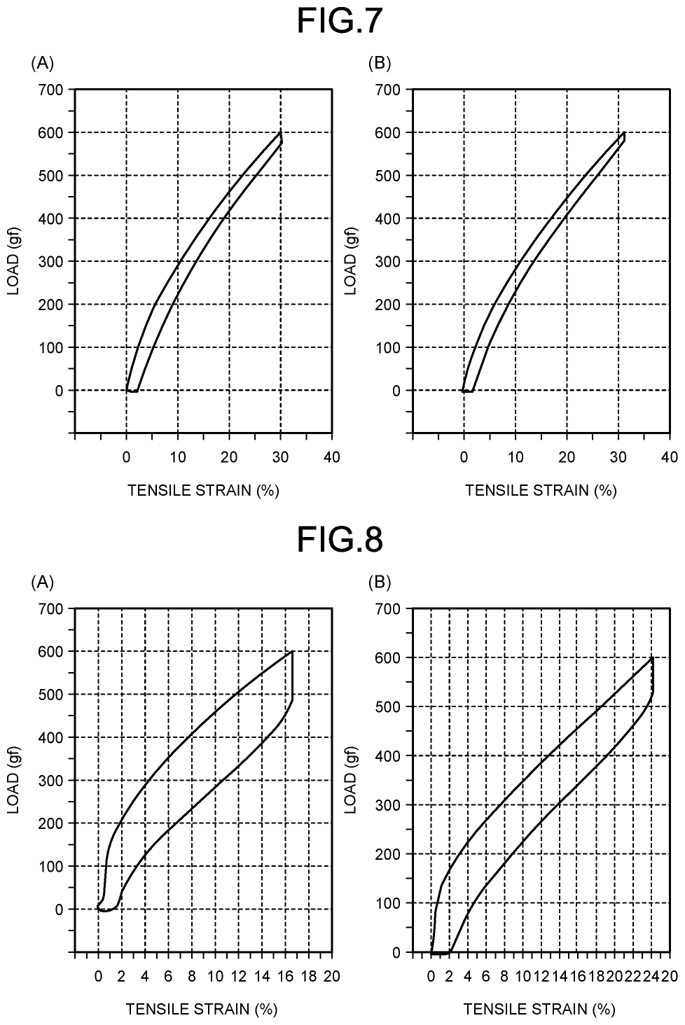

FIG. 7 illustrates the stress-strain curves before the storage (FIG. 7(A)) and after the storage (FIG. 7(B)) of the elastic body 35 that is suitable for the biological signal monitoring wear 100 according to the embodiment of the present invention. FIG. 7 illustrates the stress-strain curves before and after the storage of the flat rubber (LY-40; manufactured by Kitani Co., Ltd.), which was used as the elastic body 35 in the embodiment of the present invention, for 10 days under normal temperature and humidity condition with the expansion rate thereof kept at 30%. The stress-strain curve was measured by the method in accordance with the D-method (without repetition) in the article 8.16.2 of JIS L1096 (2015 version). The measurement instrument MODEL 5566 (manufactured by Instron Japan Co., Ltd.) was used here. The sample size with the width of 4 cm and the length of 30 cm was used with the test length of 20 cm and the pulling speed of 30 cm/minute. In FIG. 7, there was no significant change in the shapes of the stress-strain curves before and after the storage of the elastic body 35; the load with the pulling stress of 30% in a longitudinal direction of the flat rubber was 600 gf (5.9 N) before the storage and 580 gf (5.7 N) after the storage for 10 days, indicating that there was only a slight decrease by the storage.

FIG. 8 illustrates the stress-strain curves before the storage (FIG. 8(A)) and after the storage (FIG. 8(B)) of the elastic body that is unsuitable for the biological signal monitoring wear 100 according to the embodiment of the present invention. FIG. 8 illustrates the stress-strain curves before and after the storage of the flat rubber (YI-30M; manufactured by Kitani Co., Ltd.), which was used as the elastic body unsuitable for the present invention and was stored similarly to FIG. 7 for 10 days under normal temperature and humidity condition with the expansion rate thereof kept at 30%. These conditions are the same as those of FIG. 7 except that the sample size thereof is changed to the width of 3 cm. As can be seen in FIG. 8, a significant change was recognized in the shapes of the stress-strain curves in the flat rubber before and after the storage thereof; the load with the expansion stress of 10% in a longitudinal direction of the flat rubber was rapidly decreased from 450 gf (4.4 N) before the storage to 350 gf (3.4 N) after the storage for 10 days.

### EXAMPLES

Next, the biological signal monitoring wear according to the present invention will be explained in detail with referring to Examples. The biological signal monitoring wear according to the present invention is not limited to these Examples.

### Comparative Example 1

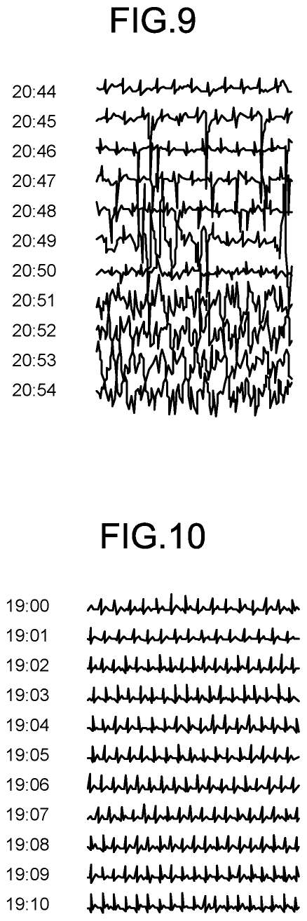

Flexible electrocardiograph cables (“hitoe” Medical Lead Wire; manufactured by Toray Medical Co., Ltd.), electrodes for electrocardiogram (“hitoe” Medical Electrode; manufactured by Toray Medical Co., Ltd.), and a Holter electrocardiograph (Kenz Cardy 303 pico+; manufactured by Suzuken Co., Ltd.) were mounted to the wear (M size) based on Patent Literature 1, and the electrocardiograms of 3 healthy male subjects were measured for a period of a half day to one day under an environment of a normal daily life. In Table 1, acquisition rates of the electrocardiogram (electrocardiogram with which the electrocardiogram analysis is possible) are described; and in FIG. 9, the electrocardiogram of the subject 1 during the time of body movement is described. For analysis of the electrocardiogram, an analysis software for the Holter electrocardiogram (Kenz Cardy Analyzer Lite; manufactured by Suzuken Co., Ltd.) was used.

### Example 1

The electrocardiograph cables, the electrodes for electrocardiogram, and the Holter electrocardiograph, which are the same as those used in Comparative Example 1, were mounted to the wear main portion 30 based on the present invention; and the electrocardiograms of the same subjects as Comparative Example 1 were measured. A force (4.4 N) generated with the rubber expansion rate of 20% was applied to the electrodes using the flat rubber (LY-40; manufactured by Kitani Co., Ltd.) having the width of 4 cm and the length of 40 cm as the elastic body 35. A 2-way tricot (polyester/polyurethane) was used in the wear main portion 30, in which the M-size that is applicable to the size length of 80 cm to 100 cm around the trunk in the subject's solar plexus portion was used. The electrocardiograms were analyzed in the same way as Comparative Example 1. The acquisition rates obtained are described in Table 1, and the electrocardiogram of the subject 1 during the time of body movement is described in FIG. 10.

As can be seen in Table 1, in Comparative Example 1, the electrocardiogram acquisition rate of 90% could not be reached in any of the subjects. On the other hand, in Example 1, the electrocardiogram acquisition rate of almost 100% could be obtained. As can be seen in FIG. 9, in Comparative Example 1, many noises are recognized in the electrocardiogram during the time of body movement; on the other hand, in Example 1, a stable electrocardiogram could be obtained even during the time of body movement.

TABLE 1

| | Comparative Example 1 | | | | Example 1 | | | |
|---|---|---|---|---|---|---|---|---|
| | | | | Acquisition | | | | Acquisition |
| | | | Total | rate of | | | Total | rate of |
| | Measurement | | heartbeat | electrocardiogram | Measurement | | heartbeat | electrocardiogram |
| Subject | Period | | number | (%) | Period | | number | (%) |
| 1 | 11 | Hours | 29187 | 70.7 | 14 | Hours | 58563 | 99.7 |
| 2 | 11 | Hours | 44425 | 87.9 | 24 | Hours | 83930 | 100 |
| | 40 | minutes | | | | | | |
| 3 | 19 | Hours | 64188 | 89.2 | 18 | Hours | 76654 | 99.9 |

| | |
|---|---|
| 40 | minutes |

### Comparative Example 2

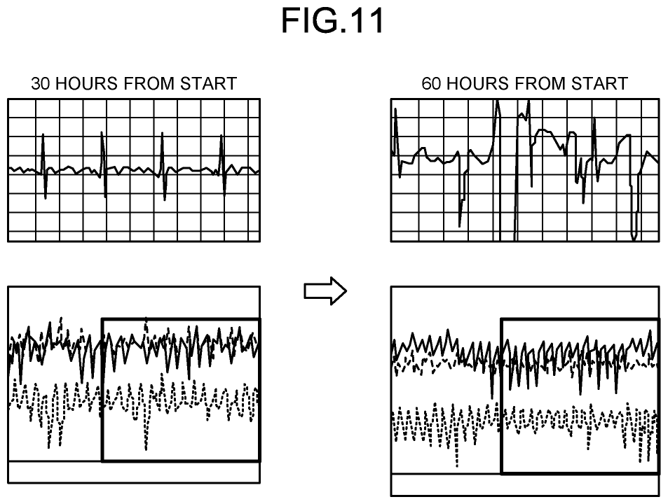

Electrocardiograph cables (“hitoe” Medical Lead Wire II; manufactured by Toray Medical Co., Ltd.), electrodes for electrocardiogram (“hitoe” Medical Electrode II; manufactured by Toray Medical Co., Ltd.), and a Holter electrocardiograph (EV-301; manufactured by Parama Tech Co., Ltd.) were mounted to the biological signal monitoring wear having a band-like structure based on Patent Literature 3; and the electrocardiogram of a healthy male subject was measured for a period of 3 days under an environment of a normal daily life. A commercially available belt formed of a polyurethane elastic fiber was used as a belt. Analysis of the electrocardiogram was carried out by using an analysis software for the Holter electrocardiogram (NEY-HEA 3000 (long time Holter electrocardiogram analysis viewer); manufactured by Nexis Co., Ltd.). FIG. 11 illustrates the swing width that indicates the electrocardiogram and a state of the body movement after 30 hours and 60 hours from the start of the measurement, respectively, measured by a three-dimensional acceleration meter.

### Example 2

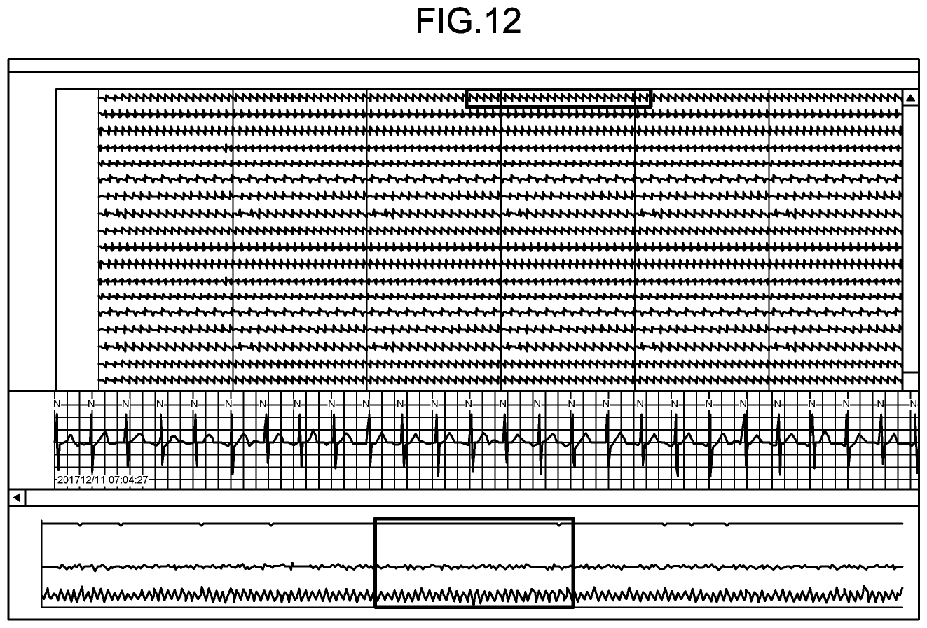

The electrocardiograph cables, the electrodes for electrocardiogram, and the Holter electrocardiograph, which are the same as those used in Comparative Example 2, were mounted to the wear main portion 30 based on the present invention; and the electrocardiogram of the same subject as Comparative Example 2 were measured for 14 days. A force (5.9 N) obtained by the rubber expansion rate of 30% using, as the elastic body to the electrodes, the flat rubber (LY-40; manufactured by Kitani Co., Ltd.) having the width of 4 cm and the length of 40 cm was applied to the electrodes. A 2-way tricot (polyester/polyurethane) was used in the wear main portion 30, in which the M-size that is applicable to the size length of 80 cm to 100 cm around the trunk in the subject's solar plexus portion was used. The electrocardiogram was analyzed in the same way as Comparative Example 2. In FIG. 12, the compressed data of the electrocardiogram, the enlarged wave form, and the body movement data after 14 days from the start of the measurement are described.

As can be seen in FIG. 11, in Comparative Example 2, a stable electrocardiogram was obtained after 30 hours from the start of the measurement, but after 60 hours from the start of the measurement, the electrocardiogram was disturbed during the time of body movement so that the electrocardiogram stable enough for the analysis thereof was not obtained. In Example 2, as can be seen in FIG. 12, the electrocardiogram stable enough for the analysis thereof was obtained even after 14 days from the start of the measurement.

### Example 3

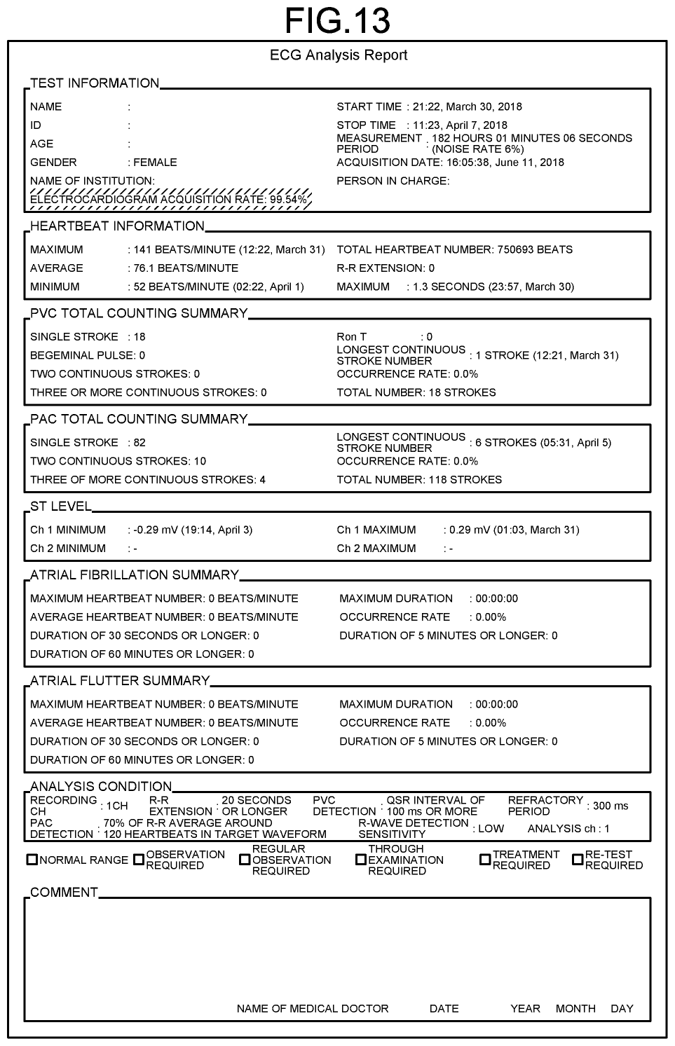

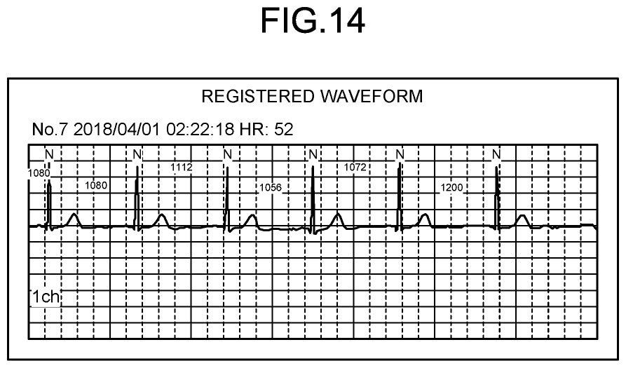

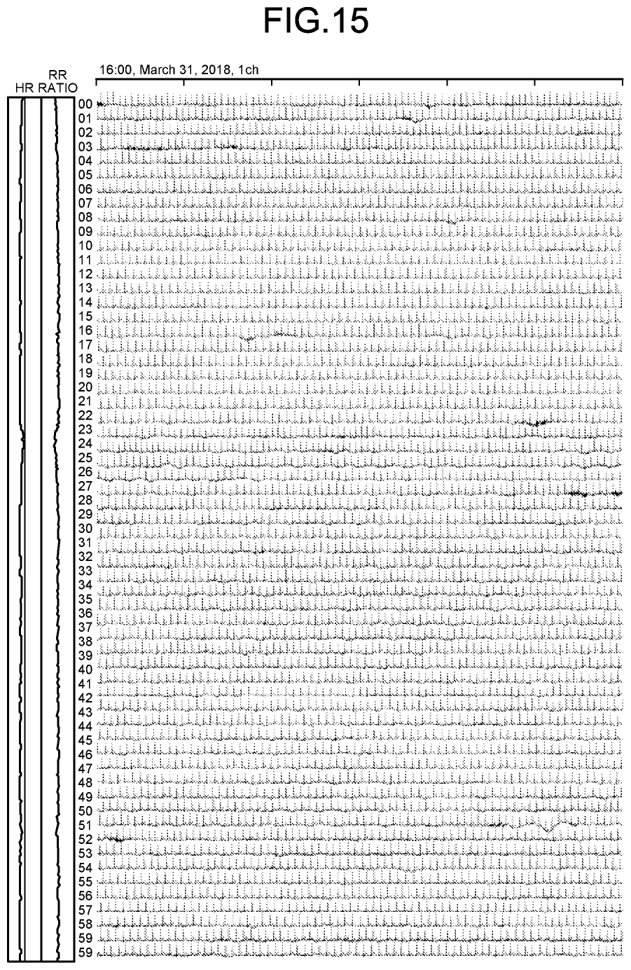

The Holter electrocardiograph (EV-301; manufactured by Parama Tech Co., Ltd.) was mounted to the biological signal monitoring wear based on the present invention; and the electrocardiogram of a healthy female subject was measured for a period of 8 days under an environment of a normal daily life. The electrocardiograph cables of “hitoe” Medical Lead Wire II manufactured by Toray Medical Co., Ltd., and the electrodes for electrocardiogram of “hitoe” Medical Electrode II manufactured by Toray Medical Co., Ltd. were used. The flat rubber (LY-40; manufactured by Kitani Co., Ltd.) having the width of 4 cm and the length of 30 cm was used as the elastic body. The two-way tricot (polyester/polyurethane) was used for the wear main portion 30, and the wear size of which was the S-size (the length around the trunk in the subject's solar plexus portion is 60 cm to 80 cm). The expansion rate of the flat rubber was 30%, and the force thereby obtained was 5.9 N. The software used in analysis of the electrocardiogram was the long time Holter electrocardiogram analysis viewer NEY-HEA 3000, manufactured by Nexis Co., Ltd. FIG. 13 is a front cover portion of the electrocardiogram analysis report, the summary of which is described in this cover portion. The measurement results regarding the heartbeat information, PVC (premature ventricular contraction), PAC (premature atrial contraction), ST level, atrial fibrillation, and atrial flutter are summarized in one sheet. The acquisition rate of the electrocardiogram obtained for the measurement period of 182 hours was 99.5%. From this, it can be seen that the electrocardiogram stable enough for analysis thereof for a long period of time could be obtained. FIG. 14 describes one registered waveform. Typical sinus rhythm was obtained, in which the P wave, the QRS wave, and the T wave can be clearly read. FIG. 15 is a so-called compressed waveform, indicating a summary of the electrocardiogram measured for a period of 1 hour in one sheet.

### REFERENCE SIGNS LIST

10 Electrocardiograph
20, 21, 22 Electrode
30 Wear main portion
31 Front body
32 Back body
33 Shoulder strap
34 Trunk portion
35 Elastic body
36 Side tab
37 Connector
40 B-face of plane fastener
41 Stitch
100 Biological signal monitoring wear

1. A biological signal monitoring wear comprising:
a biological signal measurement device;
two or more electrodes contacting to skin; and
a wear main portion in which an electrically conductive body configured to connect between the biological signal measurement device and the electrodes is supported on a cloth member of the wear main portion, and an elastic body having a length in a range of 30% to 60% inclusive relative to a length around a trunk in a subject's solar plexus portion is fixed to a trunk portion in the cloth member.

2. The biological signal monitoring wear according to claim 1, wherein a force to expand the elastic body by 30% in a longitudinal direction of the elastic body is in a range of 3 N to 9 N inclusive.

3. The biological signal monitoring wear according to claim 1, wherein a force to expand the elastic body by 20% in a longitudinal direction of the elastic body is in a range of 2 N to 6 N inclusive.

4. The biological signal monitoring wear according to claim 1, wherein when the elastic body is stored for 10 days under a normal temperature and humidity condition with an expansion rate of the elastic body kept at 30% in a longitudinal direction of the elastic body, a force to expand the elastic body by 10% after the storage is 80% or more relative to the force required before the storage.

5. The biological signal monitoring wear according to claim 1, further comprising a size-adjustment functioning portion to adjust an expansion rate of the elastic body in a subject having a different length around a trunk in a solar plexus portion.

6. The biological signal monitoring wear according to claim 5, wherein the size-adjustment functioning portion has tick marks in a size-adjustment portion in the size-adjustment functioning portion.

7. The biological signal monitoring wear according to claim 1, wherein the wear main portion comprises a front body, a back body, and at least one shoulder strap configured to connect between the front body and the back body.

8. The biological signal monitoring wear according to claim 7, wherein the front body and the back body are a bib-type wear in which at least one side of the bib-type wear is cut and separated to each other.

9. The biological signal monitoring wear according to claim 1, wherein the electrodes each comprise an electrically conductive fiber.

10. The biological signal monitoring wear according to claim 1, wherein the electrodes are each formed of a nanofiber whose fiber diameter is in a range of 10 nm to 5000 nm inclusive.

11. The biological signal monitoring wear according to claim 1, wherein the electrodes each comprise an electrically conductive sheet whose adhesion strength measured with a 90-degree peel-off method in accordance with JIS-Z0237 is 200 g/20 mm or less.
